(19) United States
(12) Patent Application Publication
(10) Pub. No.: US 2010/0041691 A1
(43) Pub. Date: Feb. 18, 2010

(54) PHARMACEUTICAL COMPOSITIONS COMPRISING COMBINATIONS OF AN AMPA/KAINATE ANTAGONISTIC COMPOUND AND AN INHIBITOR OF A MULTIDRUG RESISTANCE PROTEIN

(75) Inventors: Pia Weikop, Borup (DK); Mette Bjerg Nielsen, Ballerup (DK); Lars Siim Madsen, Soro (DK); Claus Mathiesen, Vekso Sj. (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/440,422

(22) PCT Filed: Sep. 12, 2007
(86) PCT No.: PCT/EP07/59548

§ 371 Date: May 12, 2009

Related U.S. Application Data
(60) Provisional application No. 60/825,320, filed on Sep. 12, 2006.

(30) Foreign Application Priority Data
Sep. 12, 2006 (DK) PA 2006 01164

(51) Int. Cl. A61K 31/4738

(52) U.S. Cl. 514/292

(57) ABSTRACT

This invention relates to pharmaceutical compositions comprising a combination of an AMPA/kainate antagonistic compound and an inhibitor of a multidrug resistance protein and its use for combating epileptic disorders.

### TECHNICAL FIELD

This invention relates to pharmaceutical compositions comprising an AMPA/kainate antagonistic compound and an inhibitor of a multidrug resistance protein, and their use for combating CNS disorders.

### BACKGROUND ART

The central nervous system (CNS) consists of the brain and spinal cord. Disorders of this system are many, varied and frequently severe, affecting a large portion of the population. These debilitating disorders include diseases such as epilepsy and migraine; degenerative conditions such as Parkinson's disease; and psychiatric disorders such as anxiety, bipolar disorder, depression, and schizophrenia.

Epilepsy is the term used for a number of serious neurological diseases with the common feature that they involve attacks of various types of seizures, consciousness disturbances and abnormal perception function, and epilepsy is a common and devastating neurological disorder.

There are drugs on the market intended for treatment of e.g. different epilepsy syndromes. In many patients with epilepsy seizures are well-controlled with currently available anti-epileptic drugs, but a substantial proportion of the patients continue to have seizures despite carefully optimized drug treatment.

WO 98/14447 describes indole-2,3-dione-3-oxime derivatives having AMPA/kainite antagonistic properties, being capable of antagonising the effect of excitatory amino acids and thus useful for combating disorders that are responsive to excitatory amino acid receptor antagonists, incl. epilepsy.

It has been shown that prolonged seizures, brain trauma and stroke increase the release of glutamate, which is one of the brain's neurotransmitters. It is well established that the accumulation of endogenous glutamate into the extracellular space of the central nervous system (CNS) is associated with many acute and chronic neurodegenerative disorders such as i.a. cerebral ischaemia, epilepsy, amyotrophic lateral sclerosis, Parkinson's and Alzheimer's disease, and AMPA/kainate antagonistic compounds are currently under development for use i.a. as antiepileptic drugs.

P-glycoprotein (P-gp) is a protein considered capable of reducing the accumulation of drugs in cells by pumping them out, and overexpression of P-gp is believed to play a significant role in pharmacoresistance by preventing e.g. the antiepileptic drugs from reaching their targets in the brain. Moreover, glutamate has been found to stimulate the expression and activity of P-gp in endothelial cells making up the blood-brain barrier (BBB), so transport of P-gp substrates in BBB appears to be affected during ischemic and anoxic injury.

In this respect van Vliet et al. [van Vliet E A, van Schaik R, Edelbroek P M, Redeker S, Aronica E, Wadman W J, Marchi N, Vezzani A and Gorter J A; Epilepsia 2006 47 (4) 672-680] have found that inhibition of P-gp with Tariquidar improves seizure control in phenytoin-treated chronic epileptic rats.

Moreover WO 2006/062683 describes a method for the treatment of epilepsy comprising administration of an agent that interferes with glutamate, aspartate and/or ATP-release from astrocytes.

### SUMMARY OF THE INVENTION

Investigations carried out by the inventors have revealed that a certain group of AMPA/kainite antagonistic compounds happens to be substrates of the multidrug resistance protein P-glycoprotein, which finding has lead to a particularly useful combination therapy residing in a the use of an AMPA/kainate antagonistic compound and an inhibitor of multidrug resistance protein P-glycoprotein for combating certain neurodegenerative disorders, and in particular for obtaining seizure control.

Accordingly, in its first aspect the invention provides pharmaceutical compositions comprising a therapeutically effective amount of (i) an AMPA/kainate antagonistic compound of Formula Ia or Ib

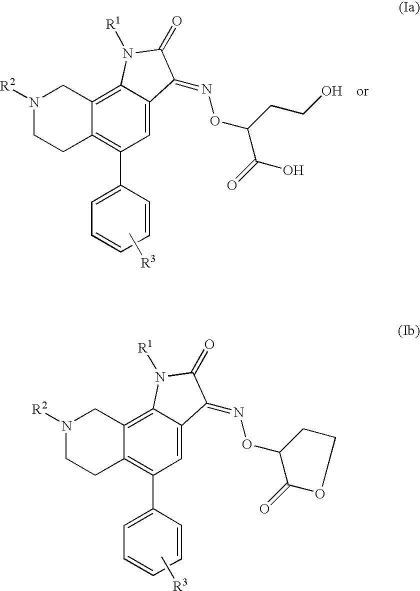

wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; and (ii) at least one inhibitor of the multidrug resistance protein P-glycoprotein; isomers thereof or a mixture of their isomers, or pharmaceutically-acceptable addition salts thereof, together with one or more adjuvants, excipients, carriers and/or diluents.

Viewed from another aspect the invention relates to the use of a combination of (i) an AMPA/kainate antagonistic compound of Formula Ia or Ib

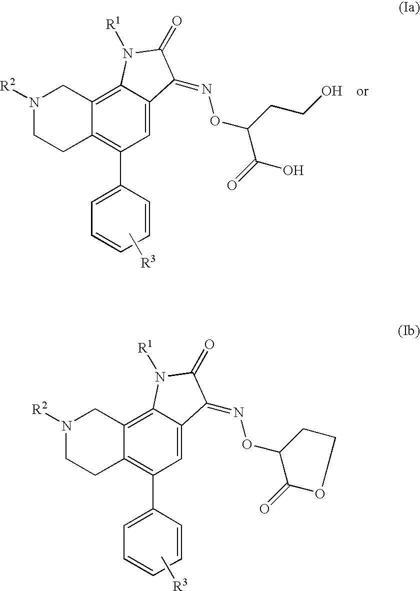

wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; and (ii) at least one inhibitor of a multidrug resistance protein; isomers thereof or a mixture of their isomers, or pharmaceutically-acceptable addition salts thereof, for the manufacture of a medicament for the treatment, prevention or alleviation of a disease or a disorder or a condition of a mammal, including a human, which disease, disorder or condition is responsive to blockade of the AMPA (α-amino-3-hydroxy-5-methylisoxazole-4-propionic acid) subtype of neuronal glutamate receptors by glutamic and/or aspartic acid receptor antagonists.

In a third aspect the invention provides a kit of parts comprising at least two separate unit dosage forms (A) an AMPA/kainate antagonistic compound of Formula Ia or Ib

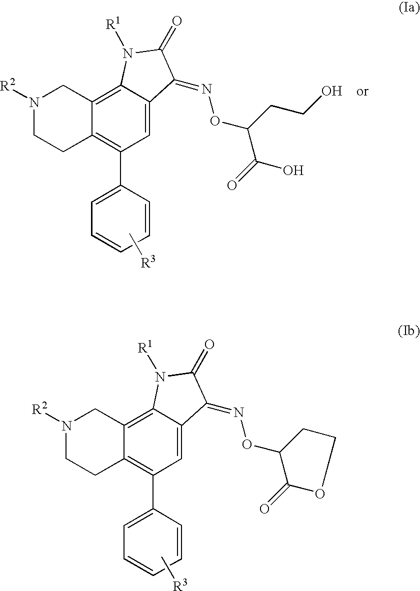

wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; an isomer thereof or a mixture of its isomers, or a pharmaceutically-acceptable addition salt thereof, together with one or more adjuvants, excipients, carriers and/or diluents; and (B) at least one inhibitor of the multidrug resistance protein P-glycoprotein; an isomer thereof or a mixture of its isomers, or a pharmaceutically-acceptable addition salt thereof, together with one or more adjuvants, excipients, carriers and/or diluents; and optionally (C) instructions for the simultaneous, sequential or separate administration of the AMPA/kainate antagonistic compound of (A) and the inhibitor of the multidrug resistance protein P-glycoprotein of (B) to a patient in need thereof.

In a further aspect the invention provides methods of treatment, prevention or alleviation of a disease or a disorder or a condition of a living animal body, including a human, which disorder, disease or condition is responsive to blockade of the AMPA (α-amino-3-hydroxy-5-methylisoxazole-4-propionic acid) subtype of neuronal glutamate receptors by glutamic and/or aspartic acid receptor antagonists, which method comprises the step of administering to such a living animal body in need thereof, a therapeutically effective amount of a combination of (i) an AMPA/kainate antagonistic compound of Formula Ia or Ib

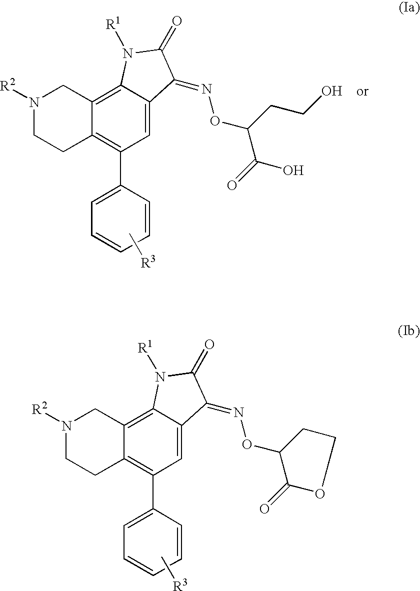

wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; and (ii) at least one inhibitor of a multidrug resistance protein; isomers thereof or a mixture of their isomers, or pharmaceutically-acceptable addition salts thereof.

Other objects of the invention will be apparent to the person skilled in the art from the following detailed description and examples.

### DETAILED DISCLOSURE OF THE INVENTION

The present invention relates to the use of a particular combination of (i) an AMPA/kainate antagonistic compound as defined below, and (ii) at least one inhibitor of a multidrug resistance protein as also defined below, for combating certain CNS diseases, as described in more details below.

### AMPA/Kainate Antagonistic Compounds

The AMPA/kainate antagonistic compounds for use according to the invention may be characterised as a compound of Formula Ia or Ib

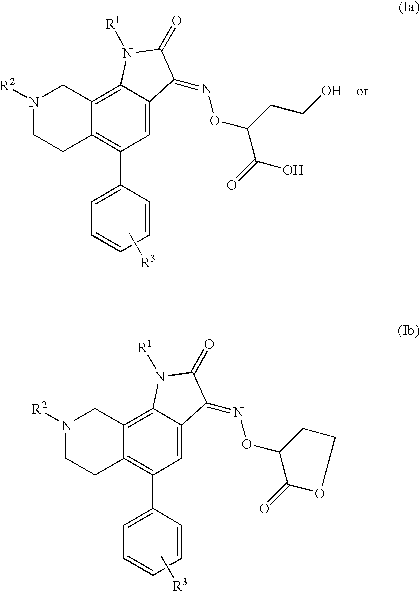

wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; isomers thereof or a mixture of their isomers, or pharmaceutically-acceptable addition salts thereof, together with one or more adjuvants, excipients, carriers and/or diluents.

In a preferred embodiment the AMPA/kainate antagonistic compound for use according to the invention is a compound of Formula Ia or Ib, wherein R¹ represents hydrogen or alkyl.

In a more preferred embodiment the AMPA/kainate antagonistic compound for use according to the invention is a compound of Formula Ia or Ib, wherein R² represent hydrogen or alkyl, and in particular methyl.

In another more preferred embodiment the AMPA/kainate antagonistic compound for use according to the invention is a compound of Formula Ia or Ib, wherein R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl, and in particular N,N-dimethyl-sulfonyl.

In a third more preferred embodiment the AMPA/kainate antagonistic compound for use according to the invention is a compound of Formula Ia or Ib, wherein R¹ represents hydrogen; R² represent alkyl, and in particular methyl; and R³ represents N,N-dialkyl-sulfonyl, and in particular N,N-dimethyl-sulfonyl.

In a fourth more preferred embodiment the AMPA/kainate antagonistic compound for use according to the invention is a compound of Formula Ia, wherein R¹ represents hydrogen; R² represent alkyl, and in particular methyl; and R³ represents N,N-dialkyl-sulfonyl, and in particular N,N-dimethyl-sulfonyl.

In a fifth more preferred embodiment the AMPA/kainate antagonistic compound for use according to the invention is a compound of Formula Ia or Ib,

wherein R¹ represents hydrogen; R² represent methyl; and R³ represents N,N-dimethyl-sulfonyl.

In a sixth more preferred embodiment the AMPA/kainate antagonistic compound for use according to the invention is a compound of Formula Ia, wherein R¹ represents hydrogen; R² represent methyl; and R³ represents N,N-dimethyl-sulfonyl.

In a most preferred embodiment the AMPA/kainate antagonistic compound for use according to the invention is

8-Methyl-5-(4-(N,N-dimethylsulfamoyl)phenyl)-6,7,8,9-tetrahydro-1H-pyrrolo[3,2h]-isoqinoline-2,3-dione-3-O-(4-hydroxybutyric acid-2-yl)oxime, an isomer thereof or a mixture of its isomers, or a pharmaceutically-acceptable addition salt thereof; or

(R)-2-[5-(4-Dimethylsulfamoyl-phenyl)-8-methyl-2-oxo-1,2,6,7,8,9-hexahydro-pyrrolo[3,2-h]isoquinolin-(3Z)-ylideneaminooxy]-4-hydroxy-butyric acid; or

(S)-2-[5-(4-Dimethylsulfamoyl-phenyl)-8-methyl-2-oxo-1,2,6,7,8,9-hexahydro-pyrrolo[3,2-h]isoquinolin-(3Z)-ylideneaminooxy]-4-hydroxy-butyric acid;

or a pharmaceutically-acceptable addition salt thereof.

### Definition of Substituents

In the context of this invention alkyl designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention alkoxy designates an “alkyl-O-” group, wherein alkyl is as defined above. Examples of preferred alkoxy groups of the invention include methoxy and ethoxy.

In the context of this invention halo represents fluoro, chloro, bromo or iodo.

### Isomers

It will be appreciated by those skilled in the art that the compounds for use according to the present invention may exist in different stereoisomeric forms, including enantiomers, diastereomers, as well as geometric isomers (cis-trans isomers). The invention includes the use of any such isomers and any mixtures thereof, including racemic mixtures.

### Pharmaceutically Acceptable Salts

The compounds for use according to the invention may be provided in any form suitable for the intended administration. Suitable forms include pharmaceutically (i.e. physiologically) acceptable salts, and pre- or prodrug forms of the compound for use according to the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a compound for use according to the invention include, without limitation, the sodium, the potassium, the calcium, the magnesium, the zinc, the aluminium, the lithium, the choline, the lysine, and the ammonium salt, and the like, of a compound for use according to the invention containing an anionic group. Such cationic salts may be formed by procedures well known and described in the art.

### Multidrug Resistance Protein Inhibitors

The multidrug resistance protein inhibitor for use according to the invention preferably is an inhibitor of P-glycoprotein, also designated P-gp, MDR1 (multidrug resistance 1) or ABCB1 (ATP-binding cassette B1).

In a more preferred embodiment the inhibitor of the P-glycoprotein (P-gp) inhibiting compound for use according to the invention is selected from Tariquidar (also designated TQD or XR-9576), Zosuquidar (also designated LY-335979), Laniquidar (also designated R-101933), OC-144-093, ONT-093, Dexverapamil, Dexnuguldipine, Valpodar (also designated PSC833), Biricodar (also designated VX-710), Verapamil, Cyclosporin A, and Tamoxifen.

In another more preferred embodiment the inhibitor of the P-glycoprotein (P-gp) inhibiting compound for use according to the invention is selected from Tariquidar, Zosuquidar, Laniquidar, OC-144-093, ONT-093, Dexverapamil, Dexnuguldipine, Valpodar and Biricodar.

In a third more preferred embodiment the inhibitor of the P-glycoprotein (P-gp) inhibiting compound for use according to the invention is selected from Tariquidar, Zosuquidar, Laniquidar, OC-144-093, ONT-093, Dexverapamil, Dexnuguldipine, Valpodar, Biricodar, Verapamil, Cyclosporin A, and Tamoxifen.

In a fourth more preferred embodiment the inhibitor of the P-glycoprotein (P-gp) inhibiting compound for use according to the invention is selected from Tariquidar, Zosuquidar, Laniquidar, OC-144-093, ONT-093, Dexverapamil, Dexnuguldipine, Valpodar, and Biricodar.

In a fifth more preferred embodiment the inhibitor of the P-glycoprotein (P-gp) inhibiting compound for use according to the invention is selected from Tariquidar, Zosuquidar, Laniquidar, OC-144-093, ONT-093, and Verapamil.

In a sixth more preferred embodiment the inhibitor of the P-glycoprotein (P-gp) inhibiting compound for use according to the invention is selected from Tariquidar, Zosuquidar, Laniquidar, OC-144-093 and ONT-093.

In a seventh more preferred embodiment the inhibitor of the P-glycoprotein (P-gp) inhibiting compound for use according to the invention is selected from Tariquidar and Verapamil.

In a most preferred embodiment the inhibitor of the P-glycoprotein (P-gp) inhibiting compound for use according to the invention is Tariquidar (also designated TQD or XR-9576).

In another most preferred embodiment the inhibitor of the P-glycoprotein (P-gp) inhibiting compound for use according to the invention is OC-144-093.

Any combination of two or more of the embodiments described herein is considered within the scope of the present invention.

### Biological Activity

Pharmacokinetic experiments with the pharmaceutical combinations described above have shown promising for their usefulness for combating certain CNS disorders.

In a preferred embodiment the disease, disorder or condition contemplated in this respect is an ischaemic condition, in particular a cerebrovascular disorder, cerebral ischaemia, lathyrism, cerebral infarction, stroke, transient myocardial infarction, Alzheimer's disease, Huntington's diseases, Amyotropic Lateral Sclerosis (ALS), psychosis, schizophrenia, Parkinsonism, a convulsive disorder, epilepsy, anxiety, pain or migraine.

The cerebrovascular disorder may in particular be cerebral ischemia or cerebral infarction resulting from a range of conditions such as tromboembolic or haemorrhagic stroke, cerebral vasospasm, hypoglycaemia, cardiac arrest, perinatal asphyxia, anoxia such as from near-drowning, pulmonary surgery and cerebral trauma, and the drug combination for use according to the invention may be used in the treatment or prevention of traumatic brain injury, in particular ischaemic, hypoxic or anoxic brain damage, spinal cord injury, tissue ischemia and reperfusion injury in a mammal at risk for such damage.

In another preferred embodiment the disease, disorder or condition contemplated in this respect is a convusive disorder, in particular epilepsia, status epilepticus, or seizures.

In its most preferred embodiment the combination therapy is contemplated used as an antiepileptic drug combination for obtaining seizure control.

### Pharmaceutical Compositions

While the invention may be practised by the provision of one pharmaceutical compositions comprising both types of compounds, i.e. the AMPA/kainate antagonistic compound (i), and one or more inhibitors of multidrug resistance protein P-glycoprotein of (ii) (i.e. a combined pharmaceutical composition), the invention may also be practised by the provision of two or more separate pharmaceutical compositions, each comprising one of the individual substances for simultaneous, sequential or separate administration. The latter embodiment may in particular be practiced by the provision of a pharmaceutical kit of parts as describe below.

While a compounds of the invention for use in therapy may be administered in the form of the raw compound, it is preferred to introduce the active ingredient, optionally in the form of a physiologically acceptable salt, or in the form of a prodrug, in a pharmaceutical composition together with one or more adjuvants, excipients, carriers, buffers, diluents, and/or other customary pharmaceutical auxiliaries.

In a preferred embodiment, the invention provides pharmaceutical compositions comprising the compounds of the invention, or a pharmaceutically acceptable salt or derivative thereof, together with one or more pharmaceutically acceptable carriers, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition of the invention can be prepared by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Pharmaceutical Kits of Parts

According to the invention there is also provided a kit of parts comprising at least two separate unit dosage forms (A) and (B):

(A) an AMPA/kainate antagonistic compound of Formula Ia or Ib

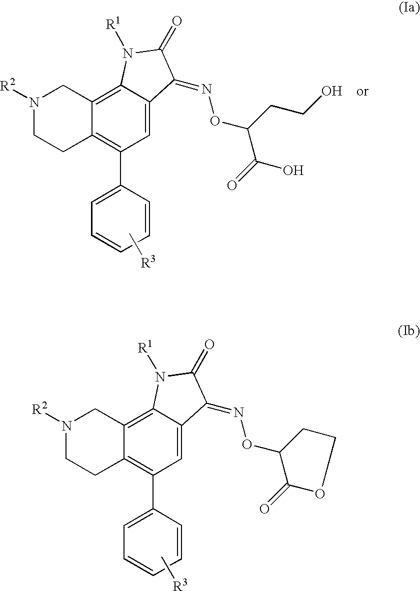

wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; an isomer thereof or a mixture of its isomers, or a pharmaceutically-acceptable addition salt thereof, together with one or more adjuvants, excipients, carriers and/or diluents; and

(B) at least one inhibitor of the multidrug resistance protein P-glycoprotein; an isomer thereof or a mixture of its isomers, or a pharmaceutically-acceptable addition salt thereof, together with one or more adjuvants, excipients, carriers and/or diluents; and optionally

(C) instructions for the simultaneous, sequential or separate administration of the AMPA/kainate antagonistic compound of (A) and the inhibitor of the multidrug resistance protein P-glycoprotein of (B) to a patient in need thereof.

The AMPA/kainate antagonistic compound for use according to the invention and the inhibitor of the multidrug resistance protein P-glycoprotein for use according to the invention may preferably be provided in a form that is suitable for administration in conjunction with the other. This is intended to include instances where one or the other of two formulations may be administered (optionally repeatedly) prior to, after, and/or at the same time as administration with the other component.

Also, the AMPA/kainate antagonistic compound for use according to the invention and the inhibitor of the multidrug resistance protein P-glycoprotein for use according to the invention may be administered in a combined form, or separately or separately and sequentially, wherein the sequential administration is close in time or remote in time. This may in particular include that two formulations are administered (optionally repeatedly) sufficiently closely in time for there to be a beneficial effect for the patient, that is greater over the course of the treatment of the relevant condition than if either of the two formulations are administered (optionally repeatedly) alone, in the absence of the other formulation, over the same course of treatment. Determination of whether a combination provides a greater beneficial effect in respect of, and over the course of treatment of, a particular condition, will depend upon the condition to be treated or prevented, but may be achieved routinely by the person skilled in the art.

When used in this context, the terms “administered simultaneously” and “administered at the same time as” include that individual doses of the AMPA/kainate antagonistic compound and the inhibitor of the multidrug resistance protein P-glycoprotein are administered within 48 hours, e.g. 24 hours, of each other.

Bringing the two components into association with each other, includes that components (A) and (B) may be provided as separate formulations (i.e. independently of one another), which are subsequently brought together for use in conjunction with each other in combination therapy; or packaged and presented together as separate components of a “combination pack” for use in conjunction with each other in combination therapy.

### Methods of Preparation and Availability

The compounds for use according to the invention are known in the art and may be prepared by conventional methods for chemical synthesis.

AMPA/kainate antagonistic compounds of Formula Ia or Ib are known from i.a. International Patent Publication WO 98/14447.

Inhibitors of P-gp have been described by e.g. Thomas et al. [Hilary Thomas & Helen M. Coley; Cancer Control 2003 10 (2) 159-165].

Tariquidar (also designated TQD or XR-9576) is known from e.g. WO 9817648 and WO 2003095447.

Zosuquidar (also designated LY-335979) is known from e.g. WO 9424107, WO 0075121 and WO 0075132.

Laniquidar (also designated R-101933) is known from e.g. WO 9734894 and WO 9913871.

OC-144-093 is known from e.g. WO 9902155.

### Methods of Therapy

In another aspect the invention provides methods for the treatment, prevention or alleviation of diseases or disorders or conditions of living animal bodies, including humans, which diseases, disorders or conditions are responsive to blockade of the AMPA (α-amino-3-hydroxy-5-methylisoxazole-4-propionic acid) subtype of neuronal glutamate receptors by glutamic and/or aspartic acid receptor antagonists. The method of the invention comprises the step of administering to such a living animal body in need thereof, a therapeutically effective amount of a combination of

(i) an AMPA/kainate antagonistic compound of Formula Ia or Ib

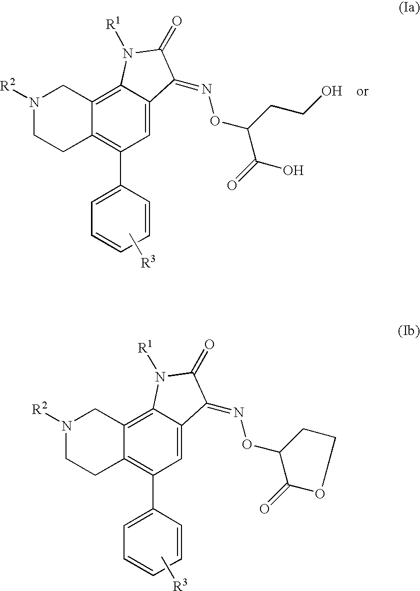

wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; and (ii) at least one inhibitor of a multidrug resistance protein; isomers thereof or a mixture of their isomers, or pharmaceutically-acceptable addition salts thereof.

In yet another aspect the invention relates to methods for the treatment, prevention or alleviation of diseases or disorders or conditions of living animal bodies, including humans, which diseases, disorders or conditions are responsive to blockade of the AMPA (α-amino-3-hydroxy-5-methylisoxazole-4-propionic acid) subtype of neuronal glutamate receptors by glutamic and/or aspartic acid receptor antagonists, which method comprises administering to the patient a therapeutically effective total amount of (i) an AMPA/kainate antagonistic compound of Formula Ia or Ib

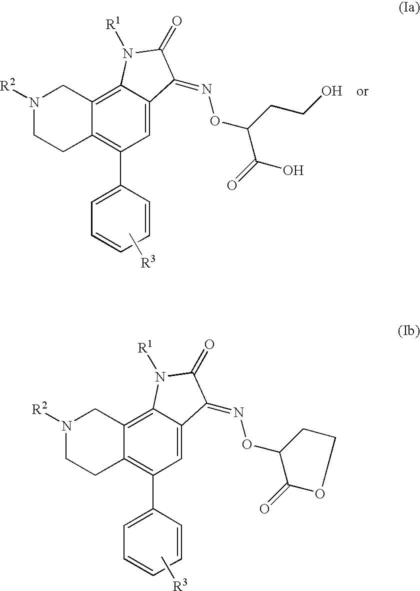

wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; and (ii) at least one inhibitor of the multidrug resistance protein P-glycoprotein; isomers thereof or a mixture of their isomers, or pharmaceutically-acceptable addition salts thereof, together with one or more adjuvants, excipients, carriers and/or diluents.

In the context of this invention the term “treatment” covers treatment, prevention, prophylaxis or alleviation, and the term “disease” covers illnesses, diseases, disorders and conditions related to the disease in question.

The preferred medical indications contemplated according to the invention are those stated above.

It is at present contemplated that suitable dosage ranges are within 0.1 to 1000 milligrams daily, preferably 10 to 500 milligrams daily, and more preferred of from 30 to 100 milligrams daily, dependent as usual upon the exact mode of administration, form in which administered, the indication toward which the administration is directed, the subject involved, the body weight of the subject involved, and further the preference and experience of the physician or veterinarian in charge.

### BRIEF DESCRIPTION OF THE DRAWINGS

The present invention is further illustrated by reference to the accompanying drawing, in which:

FIG. 1 shows the concentration of 8-Methyl-5-(4-(N,N-dimethyl-sulfamoyl)phenyl)-6,7,8,9-tetrahydro-1H-pyrrolo[3,2h]-isoqinoline-2,3-dione-3-O-(4-hydroxybutyric acid-2-yl)oxime (Compound A), in brain tissue from mice after 10 mg/kg intravenous administration [□ represents the administration of Compound A+Verapamil; and ∘ represents the administration of Compound A, only];

FIG. 2 shows the concentration of 8-Methyl-5-(4-(N,N-dimethyl-sulfamoyl)phenyl)-6,7,8,9-tetrahydro-1H-pyrrolo[3,2h]-isoqinoline-2,3-dione-3-O-(4-hydroxybutyric acid-2-yl)oxime (Compound A), in plasma from mice after 10 mg/kg intravenous administration [□ represents the administration of Compound A+Verapamil; and ∘ represents the administration of Compound A, only]; and

FIG. 3 shows protection (determined as %) against tonic seizures induced by 26 mA corneal stimulation in the presence of Compound A and Tariquidar (▪; ED₅₀=24 mg/kg) versus treatment with Compound A and vehicle (▴; 5% chremophor; ED₅₀=31.5 mg/kg).

### EXAMPLES

The invention is further illustrated with reference to the following examples, which are not intended to be in any way limiting to the scope of the invention as claimed.

### Example 1

### Biological Activity

In this experiment the concept of using an AMPA/kainate antagonistic compound, exemplified by 8-Methyl-5-(4-(N,N-dimethylsulfamoyl)phenyl)-6,7,8,9-tetra-hydro-1H-pyrrolo[3,2h]-isoqinoline-2,3-dione-3-O-(4-hydroxybutyric acid-2-yl)oxime (Compound A), and an inhibitor of multidrug resistance protein P-glycoprotein, exemplified by Verapamil, is demonstrated.

16 NMRI-mice were allocated into 4 groups. The animals were anesthetised in 1.5% Isuflurane and 20% O₂. Each mice were dosed with 2 μl Verapamin 2.5 mg/ml I.C.V. and as fast as possible dosed with Compound A 10 mg/kg i.v in the tail vein. At different time point (5 minutes, 15 minutes, 30 minutes and 60 minutes) after dosing the animals were killed by decapitation and blood and brain were collected. The EDTA stabilised blood was centrifugation at 4000 rpm for 20 minutes at 4° C. Plasma and brain samples were stored at −20° C. pending analysis. No clinical signs were observed.

Plasma and brain samples were analysed using LC-MS/MS (Quattro Ultimo Platinum with Acquility solvent and sample manager, Waters) in positive electrospray 10 ionisation and multiple reaction monitoring mode. The analytical column was a UPLC Acquility BEH C18 (1.7 μm) 1.0×50 mm (Waters) with gradient elution at a 0.13 ml/min flow rate. The initial mobile phase was 20% methanol/80% formic acid changed to 90% methanol/10% formic acid over a time period of 1.7 min.

Aliquots of plasma (100 μl) were precipitated with 300 μl acetonitril containing deuterized (d6) Compound A as internal standard (100 ng/ml). Samples were centrifuged for 25 minutes at 5° C., 14000 rpm and 3 μl of the supernatants were injected onto the LC-column.

Brain tissue (0.1 g) was weighed out on top of a tube containing 0.8 ml bead (1.0 mm Zirconia from BioSpec Products). 1000 μl acetonitrile/water (80/20) containing internal standard (100 ng/ml) was added to each sample before homogenisation on mini-Bead-Beater-96 (Biospec Products) for 2 minutes. Samples were centrifuged for minutes at 5° C., 14000 rpm and 3 μl supernatant was injected on to the LC-column.

Samples were quantified based on standard curves (linear, 1/×weighting), prepared in plasma and brain tissue respectively.

Concentrations of Compound A in plasma and brain tissue were compared with results from a similar 10 mg/kg i.v. Compound A study without co-administration of Verapamil.

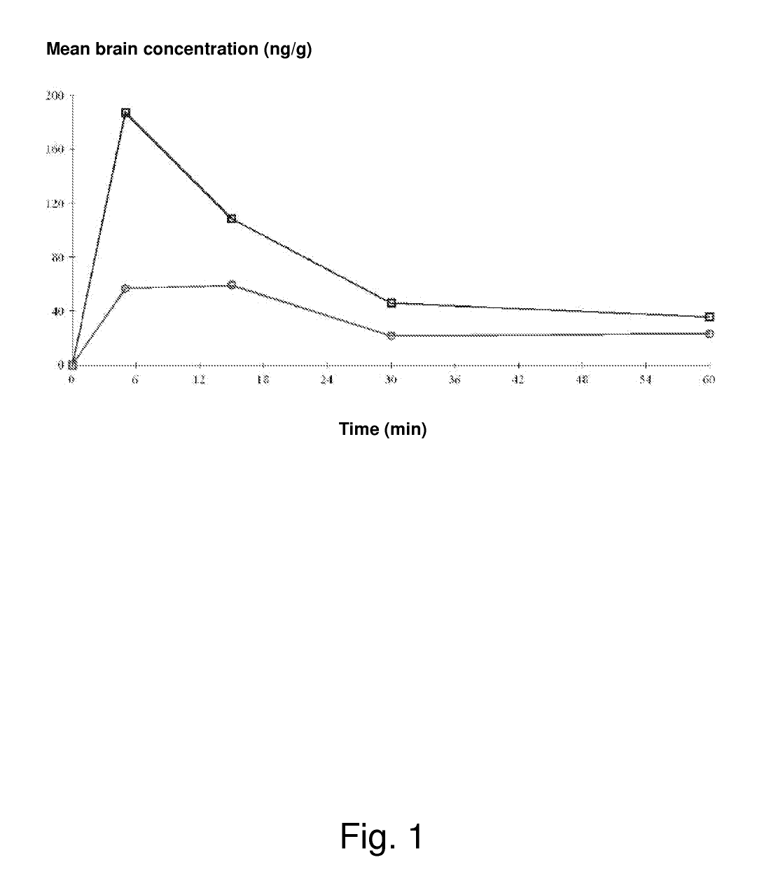

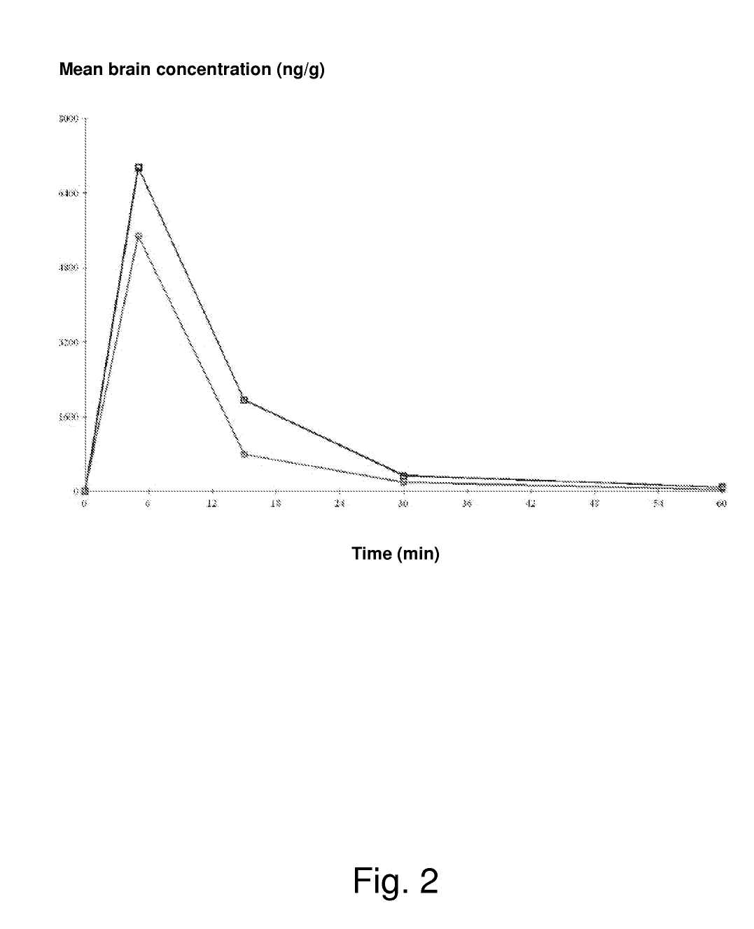

The results of this experiment are shown in FIGS. 1 and 2.

The increase in brain exposure, but little effect on plasma drug concentrations, suggests that the weak P-gp inhibitor, Verapamil blocks P-gp-mediated efflux transport of Compound A selectively from the brain.

### Example 2

### Biological Activity

In this experiment the concept of using an AMPA/kainate antagonistic compound, exemplified by 8-Methyl-5-(4-(N,N-dimethylsulfamoyl)phenyl)-6,7,8,9-tetra-hydro-1H-pyrrolo[3,2h]-isoqinoline-2,3-dione-3-O-(4-hydroxybutyric acid-2-yl)oxime (Compound A), dissolved in 0.9% NaCl, and an inhibitor of multidrug resistance protein P-glycoprotein, exemplified by Tariquidar, dissolved in 5% chremophor, is demonstrated, using the maximal electroshock threshold method.

After administration of vehicle or test substance female NMRI mice were stimulated with 26 mA for 0.3 seconds, using corneal stimulation (Rodent Shocker, Hugo Sachs Electronic, Germany). In presence of tonic seizure the mice was labeled 0% protection and if no tonic seizure was detected the mice was labeled 100% protection. Results were plotted as a function of logarithmic dose and fitted to a sigmoid-dose response (with variable slope (GraphPad Prism, ver. 4) and used to estimate the dose that would give a 50% protection from seizures (N=6/group).

Test compounds, i.e. Compound A and Tariquidar, were dosed 60 minutes (1-48 mg/kg i.v.) and 120 minutes (24 mg/kg p.o.), respectively, before electroshock test. Vehicle (5% chremophor) was dosed p.o.

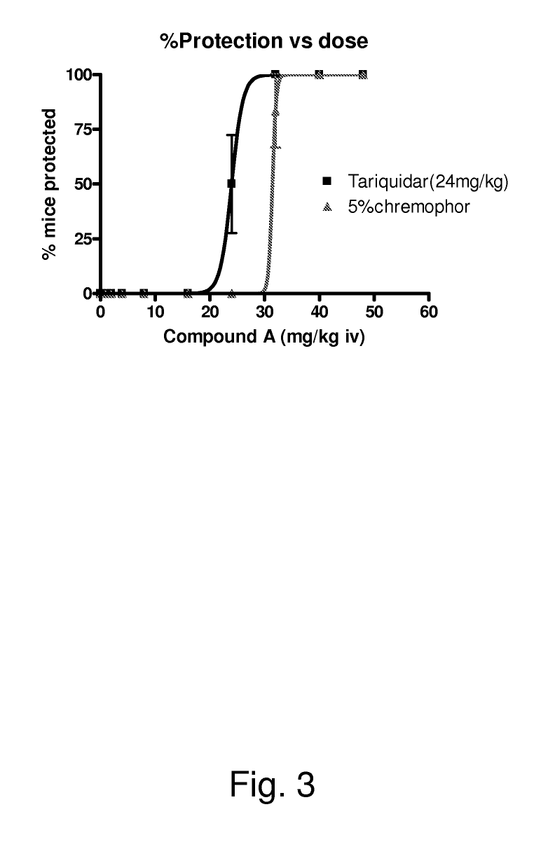

The results of this experiment are presented in FIG. 3.

This figure clearly shows that the potency of Compound A increases in presence of Tariquidar. While Compound A protected against induced tonic seizures (ED₅₀=31.5 mg/kg) the ED₅₀ value is lowered by pretreatment of Tariquidar (ED₅₀=24 mg/kg).

1. A pharmaceutical composition comprising a therapeutically effective amount of
(i) an AMPA/kainate antagonistic compound of Formula Ia or Ib 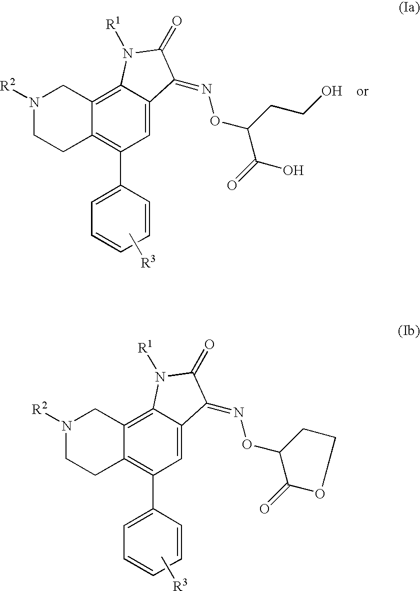
wherein
R¹ and R², independently of each other, represent hydrogen or alkyl; and
R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; and
(ii) at least one inhibitor of multidrug resistance protein P-glycoprotein;
isomers thereof or a mixture of their isomers, or pharmaceutically-acceptable addition salts thereof, together with one or more adjuvants, excipients, carriers and/or diluients.

2. The pharmaceutical composition of claim 1, wherein the AMPA/kainate antagonistic compound is a compound of Formula Ia or Ib, wherein
R¹ represents hydrogen;
R² represent alkyl; and
R³ represents N,N-dialkyl-sulfonyl.

3. The pharmaceutical composition of claim 2, wherein the AMPA/kainate antagonistic compound is a compound of Formula Ia or Ib, wherein
R¹ represents hydrogen;
R² represent methyl; and
R³ represents N,N-dimethyl-sulfonyl.

4. The pharmaceutical composition of claim 3, wherein the AMPA/kainate antagonistic compound is 8-Methyl-5-(4-(N,N-dimethylsulfamoyl)phenyl)-6,7,8,9-tetrahydro-1H-pyrrolo[3,2h]1-isoqinoline-2,3-dione-3-O-(4-hydroxybutyric acid-2-yl)oxime, an isomer thereof or a mixture of its isomers, or a pharmaceutically-acceptable addition salt thereof.

5. The pharmaceutical composition of claim 1, wherein the inhibitor of the P-glycoprotein (P-gp) inhibiting compound is selected from Tariquidar (TQD; XR-9576), Zosuquidar (LY-335979), Laniquidar (R-101933), OC-144-093, ONT-093, Dexverapamil, Dexnuguldipine, Valpodar (PSC 833), Biricodar (VX-710); Verapamil, Cyclosporin A, Tamoxifen.

6. The pharmaceutical composition of claim 5, wherein the inhibitor of the P-glycoprotein (P-gp) inhibiting compound is selected from Tariquidar (XR-9576), Zosuquidar (LY-335979), Laniquidar (R-101933), OC-144-093, ONT-093 and Verapamil.

7. The pharmaceutical composition of claim 6, wherein the inhibitor of the P-glycoprotein (P-gp) inhibiting compound is selected from Tariquidar (XR-9576) and Verapamil.

8. The pharmaceutical composition of claim 6, wherein the inhibitor of the P-glycoprotein (P-gp) inhibiting compound is Tariquidar (XR-9576).

9. (canceled)

10. The method according to claim 12, wherein the disease, disorder or condition is an ischaemic condition, in particular a cerebrovascular disorder, cerebral ischaemia, lathyrism, cerebral infarction, stroke, transient myocardial infarction, Alzheimer's disease, Huntington's diseases, Amyotropic Lateral Sclerosis (ALS), psychosis, sclizophretia, Parkinsonism, a convulsive disorder, epilepsy, anxiety, pain or migraine.

11. A kit of parts comprising at least two separate unit dosage forms (A) and (B):
(A) an AMPA/kainate antagonistic compound of Formula Ia or Ib 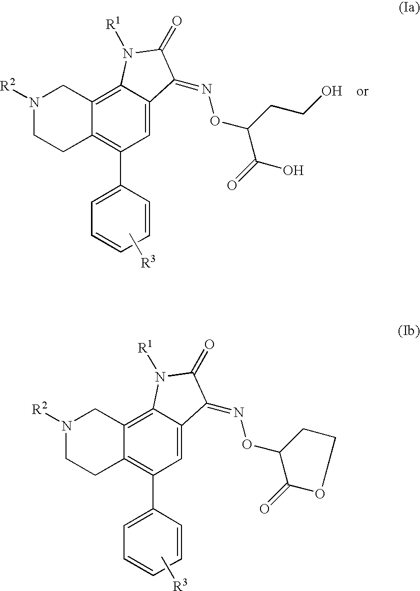
wherein R¹ and R², independently of each other, represent hydrogen or alkyl; and R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; an isomer thereof or a mixture of its isomers, or a pharmaceutically-acceptable addition salt thereof, together with one or more adjuvants, excipients, carriers and/or diluents; and
(B) at least one inhibitor of the multidrug resistance protein P-glycoprotein; an isomer thereof or a mixture of its isomers, or a pharmaceutically-acceptable addition salt thereof, together with one or more adjuvants, excipients, carriers and/or diluents; and optionally
(C) instructions for the simultaneous, sequential or separate administration of the AMPA/kainate antagonistic compound of (A) and the inhibitor of the multidrug resistance protein P-glycoprotein of (B) to a patient in need thereof.

12. A method of treatment, prevention or alleviation of a disease or a disorder or a condition of a living animal body, including a human, which disorder, disease or condition is disease or condition is responsive to blockade of the AMPA (α-amino-3-hydroxy-5-methylisoxazole-4-propionic acid) subtype of neuronal glutamate receptors by glutamic and/or aspartic acid receptor antagonists, which method comprises the step of administering to such a living animal body in need thereof, a therapeutically effective amount of a combination of
(i) an AMPA/kainate antagonistic compound of Formula Ia or Ib 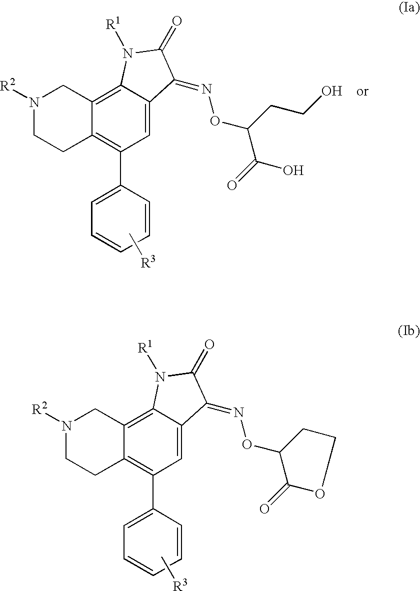
wherein
R¹ and R², independently of each other, represent hydrogen or alkyl; and
R³ represents hydrogen, alkyl, alkoxy, halo, trifluoromethyl, trifluoromethoxy, nitro, phenyl, sulfonyl, N-alkyl-sulfonyl or N,N-dialkyl-sulfonyl; and
(ii) at least one inhibitor of the multidrug resistance protein P-glycoprotein;isomers thereof or a mixture of their isomers, or pharmaceutically-acceptable addition salts thereof.
